(19) United States
(12) Patent Application Publication
(10) Pub. No.: US 2006/0194987 A1
(43) Pub. Date: Aug. 31, 2006

(54) Process for preparing tolterodine tartrate

(76) Inventors: Laszlo Zsolt Kovacs, Debrecen (HU); Csaba Szabo, Debrecen (HU); Erika Magyar Molnarne, Debrecen (HU); Claude Singer, Kfar Saba (IL)

Correspondence Address: KENYON & KENYON LLP, ONE BROADWAY, NEW YORK, NY, 10004, US

(21) Appl. No.: 11/329,922

(22) Filed: Jan. 10, 2006

Related U.S. Application Data
(60) Provisional application No. 60/642,866, filed on Jan. 10, 2005.
(60) Provisional application No. 60/690,823, filed on Jun. 14, 2005.

(51) Int. Cl. C07C 215/46

(52) U.S. Cl. 564/316

(57) ABSTRACT

The invention encompasses processes for making tolterodine tartrate.

### RELATED APPLICATIONS

This application claims the benefit of U.S. Provisional Patent Application Nos. 60/642,866 filed Jan. 10, 2005, and 60/690,823 filed Jun. 14, 2005, the contents of which are incorporated herein by reference.

### FIELD OF THE INVENTION

The invention encompasses a process for preparing Tolterodine tartrate.

### BACKGROUND OF THE INVENTION

Tolterodine is a muscarinic receptor antagonist used for the treatment of urinary urge incontinence and other symptoms of bladder over-activity. As an amine, Tolterodine forms acid addition salts when reacted with acids of sufficient strength. Pharmaceutically acceptable salts include salts of both inorganic and organic acids. The preferred pharmaceutically acceptable salt of Tolterodine is the tartrate, (R)-Tolterodine L-tartrate. The structural formula of L-(+)-tartrate of (+)-(R)-3-(2-hydroxy-5-methylphenyl)-N,N-diisopropyl-3-phenylpropylamine is shown in Formula I below.

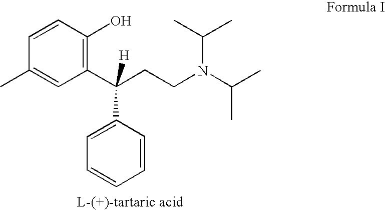

Tolterodine tartrate and a process for its preparation were first disclosed in U.S. Pat. No. 5,382,600. The '600 patent discloses the preparation of Tolterodine by deprotecting the methylether group of the diisopropyl-[3-(2-methoxymethylphenyl)-3-phenylpropyl]-amine of formula II with boron tribromide, for 2-5 days, followed by extracting Tolterodine base of formula III with a base, and then, resolving the enantiomers with L-(+)-tartaric acid in alcohol.

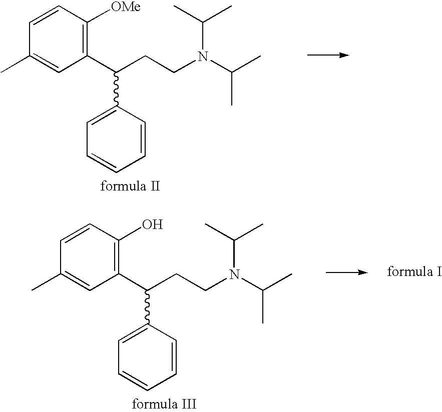

However, this method requires expensive and hazardous reagents, such as boron tribromide, reaction times of several days, and unnecessary reaction steps, such as the isolation of Tolterodine base before resolving the enantiomers of Tolterodine.

U.S. Pat. No. 5,922,914 discloses the preparation of Tolterodine by reducing a ketone using diisobutylalumnium hydride (referred to as DIBAL-H) to give compound of formula IV, which is then reduced with diisopropylamine using hydrogen over palladium on charcoal in methanol, to give Tolterodine hydrochloride salt of Formula V, followed by extracting Tolterodine base of formula III with a base, and then, resolving the enantiomers with L-(+)-tartaric acid in alcohol.

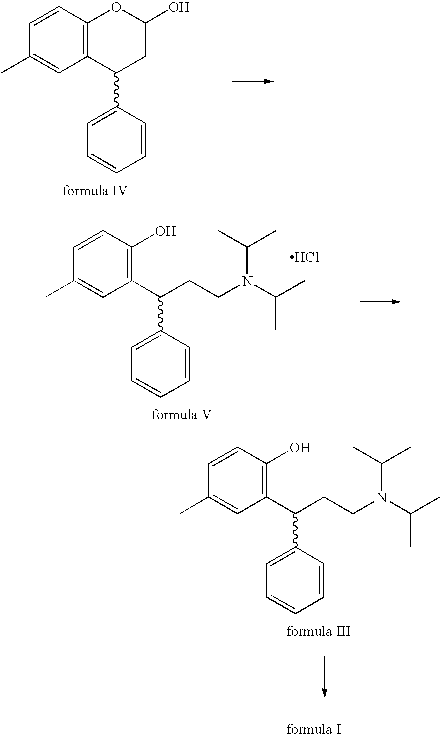

The method requires expensive and hazardous reagents, such as diisobutylaluminum hydride. In addition, the extraction of Tolterodine base of formula III, prior to the resolution step, uses dichloromethane as solvent which may not be useful on a large scale. The density of dichloromethane is higher than water; therefore, the extractions performed before the addition of L-tartaric acid can not be achieved in the same reactor.

U.S. Pat. No. 6,822,119 (WO publication No. 03/014060) prepares Tolterodine by ether cleavage comprising hydrobromic acid, boron tribromide, or catalytic hydrogenation, followed by nucleophilic substitution, leading to a product mixture that includes Tolterodine hydrobromide salt.

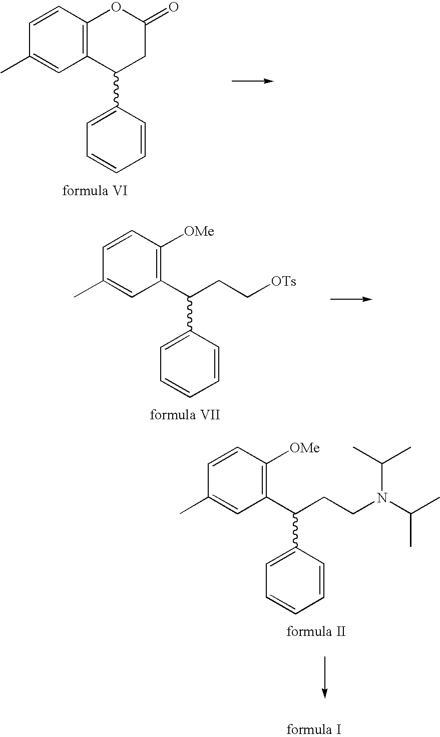

This process, however, requires about two or 3 days to complete. The '119 patent does not provide information regarding the resolution step.

Thus, there is a need in the art for a process for the preparation of Tolterodine tartrate that overcomes the limitations of the processes of the above patents.

### SUMMARY OF THE INVENTION

In one embodiment the present invention encompasses a process for the preparation of Tolterodine base of formula III,

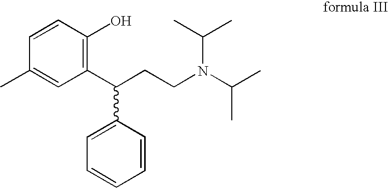

washing Tolterodine HBr with a base and a solvent selected from a group consisting of water, C₂₋₅ ester, C₂₋₆ ether, C₇₋₁₀ aromatic hydrocarbon and mixtures thereof.

In another embodiment, the present invention encompasses a process for obtaining of R-Tolterodine tartrate of formula I

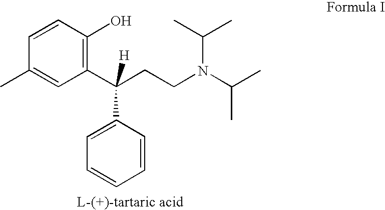

by an optical resolution process of Tolterodine base of formula III, using a solution of L-tartaric acid in a C₁₋₄ alcohol and a solution of Tolterodine base of formula III in a solvent selected from a group consisting of water, C₂₋₅ ester, C₂₋₆ ether, C₇₋₁₀ aromatic hydrocarbon and mixtures thereof.

In yet another embodiment, the present invention encompasses a process for the preparation of R-Tolterodine tartrate of formula I by cleaving the methyl ether of (N,N-diisopropyl-[3-(2-methoxy-5-methylphenyl)-3-phenylpropyl]-amine) fumarate of formula II

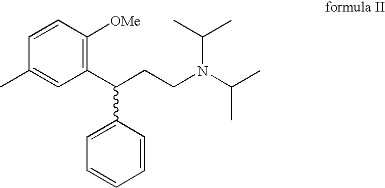

or salt thereof of Formula IIa;

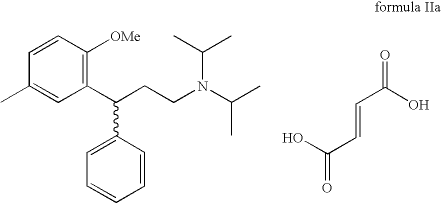

washing with a base and a solvent selected from a group consisting of water, C₂₋₅ ester, C₂₋₆ ether, C₇₋₁₀ aromatic hydrocarbon and mixtures thereof, performing an optical resolution with a solution of tartaric acid to form tolterodine tartrate; and crystallizing R-Tolterodine tartrate.

### DETAILED DESCRIPTION OF THE INVENTION

The present invention encompasses a process for the preparation of Tolterodine base of formula III,

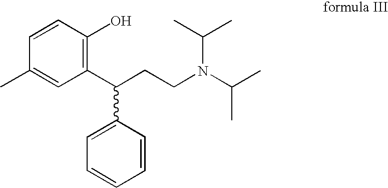

washing Tolterodine HBr with a base and a solvent selected from a group consisting of water, C₂₋₅ ester, C₂₋₆ ether, C₇₋₁₀ aromatic hydrocarbon and mixtures thereof.

Washing Tolterodine HBr may be performed by combining Tolterodine HBr with a base and a solvent selected from a group consisting of water, C₂₋₅ ester, C₂₋₆ ether, C₇₋₁₀ aromatic hydrocarbon and mixtures thereof, leads to a mixture, which is maintained, preferably, at a temperature of about 15° C. to about 30° C., more preferably, at a temperature of about 20° C. to about 25° C., for about 15 to about 30 minutes, more preferably, while stirring, prior to recovering Tolterodine base of formula III.

Preferably, the C₂₋₅ ester is either ethyl acetate or isobutyl acetate. A preferred C₂₋₆ ether is diisopropylether. Preferably, the C₇₋₁₀ aromatic hydrocarbon is toluene. More preferably, the solvent is ethyl acetate.

Preferably, the base is either an organic or inorganic base. A preferred organic base is triethylamine, diisopropylethylamine, pyridine or morpholine. Preferably, the inorganic base is added in a form of an aqueous solution. Preferably, the inorganic base is either alkali hydroxide or alkali carbonate. A preferred alkali hydroxide is either potassium hydroxide or sodium hydroxide. A preferred alkali carbonate is either potassium carbonate or sodium carbonate. More preferably, the inorganic base is potassium hydroxide. The most preferred base is potassium hydroxide.

Preferably, the base is added while stirring.

Tolterodine base of formula III may be recovered by separating the organic layer and washing with water.

The present invention further encompasses a process for obtaining of R-Tolterodine tartrate of formula I

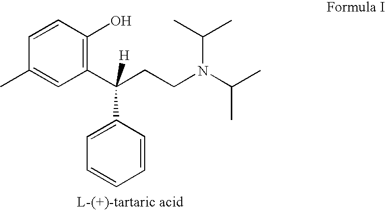

by an optical resolution process of Tolterodine base of formula III, using a solution of L-tartaric acid in a C₁₋₄ alcohol and a solution of Tolterodine base of formula III in a solvent selected from a group consisting of water, C₂₋₅ ester, C₂₋₆ ether, C₇₋₁₀ aromatic hydrocarbon and mixtures thereof.

Preferably, R-Tolterodine of Formula I is resolved directly from the solution of Tolterodine base of formula III.

Preferably, the C₁₋₄ alcohol is either ethanol or methanol, and more preferably, ethanol. Preferably, the C₂₋₅ ester is either ethyl acetate or isobutyl acetate. A preferred C₂₋₆ ether is diisopropylether. Preferably, the C₇₋₁₀ aromatic hydrocarbon is toluene. More preferably, the solvent is ethyl acetate.

The L-tartaric acid solution may be added into the Tolterodine base solution, or the Tolterodine base solution may be added to the L-tartaric acid solution. The L-tartaric acid solution may be added to the solution Tolterodine base all in one portion, meaning at one time, or over a period of time. If added over time, the addition time is preferably less than 3 hours. The reacting solutions may be combined at about room temperature to about 70° C., and preferably the solutions are combined at about room temperature, leading to a slurry.

The slurry may be cooled to 5° C. to about −5° C. for about 5 to about 17 hours. The slurry may be filtered, washed, and dried to yield Tolterodine tartrate. Preferably, the slurry is filtered using suction, washed with cold ethanol twice, and dried at 60° C. under vacuum for a period of about 3 to about 14 hours. The R-Tolterodine tartrate may be recrystallized from dry ethanol.

The process may be run stepwise or concurrently, i.e., without isolation of Tolterodine base prior to the resolution step. Preferably, the process is run concurrently before the optical resolution.

The process of the present invention for the preparation of substantially pure Tolterodine tartrate of formula I is done without requiring expensive and hazardous reagents and extensive reaction times, as compared to the product obtained by the processes of the prior art. More over, there is no need for isolation of Tolterodine base before resolving enantiomers to obtain the desired (R)-Tolterodine enantiomer, in the process of the present invention. Hazardous reagents are avoided by using, for example, anhydrous hydrobromic acid in acetic acid, which is easier to handle, for ether cleavage. Moreover, the process of the invention prepares (R)-Tolterodine enantiomer without isolating the intermediate Tolterodine base by performing the extraction of Tolterodine base and resolution of enantiomers in the same reactor. Hence, the process of the present invention is cost effective and can be adapted to industrial scale Tolterodine is prepared as described in U.S. Pat. No. 5,382,600, herein incorporated by reference.

The present invention also encompasses a process for the preparation of R-Tolterodine tartrate of formula I by cleaving the methyl ether of (N,N-diisopropyl-[3-(2-methoxy-5-methylphenyl)-3-phenylpropyl]-amine)fumarate of formula II 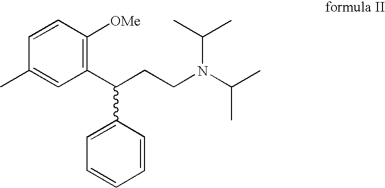
or salt thereof of formula Ia;

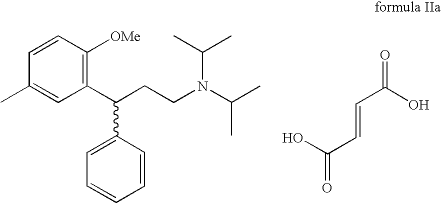

washing with a base and a solvent selected from a group consisting of water, C₂₋₅ ester, C₂₋₆ ether, C₇₋₁₀ aromatic hydrocarbon and mixtures thereof; performing an optical resolution using a solution of tartaric acid; and crystallizing R-Tolterodine tartrate.

R-Tolterodine tartrate obtained by the above process contains less than about 0.5%, and more preferably, less than 0.3% area by HPLC of total impurities.

The cleaving step of the invention is performed by treating the compound of Formula II or Ia with a solution of hydrobromic acid in acetic acid to yield a solution, which is heated to a temperature of about 70° C. to about 120° C., to give Tolterodine HBr salt, of the following structure:

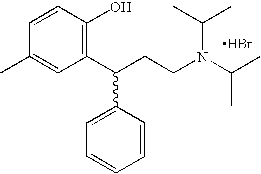

Preferably, the concentration of the hydrobromic acid in the acetic acid solution is of about 30% to about 33%.

Preferably, the reaction may be carried out at a temperature of about 75° C. to about 85° C., for about 14 hours.

Preferably, the solution is stirred during the reaction.

Tolterodine HBr salt may be recovered by cooling the solution to a temperature of about 15° C. to about 30° C., preferably, of about 20° C. to about 25° C., followed by addition of water, preferably, ice water, to form a slurry. The slurry is then cooled to a temperature of about 5° C. to about −5° C., while stirring for about a half an hour to about 24 hours, followed by filtration, washed with water, and drying, yielding Tolterodine hydrobromide. Preferably, the slurry is filtered using a suction filter, and washed with ice water twice. Drying is preferably conducted at about 60° C. to about 65° C. under vacuum.

Preferably, Tolterodine hydrobromide is obtained by the process of the invention having a purity of about 98% to about 100% area by HPLC, more preferably, of about 99% to about 100% area by HPLC.

### EXAMPLES

### Example 1

### Preparation of Tolterodine Hydrobromide

A solution was formed by combining the fumarate salt of N,N-diisopropyl-[3-(2-methoxy-5-methylphenyl)-3-phenylpropyl]-amine of structural Formula II (200 g, 0.439 mol) and HBr in acetic acid (33%, 500 ml) and stirring at about 110° C. to about 115° C. for 14 hours in a glass reactor. The solution was cooled to room temperature and ice water (2000 ml) was added, forming a slurry. The slurry was cooled to 5° C.±5° C. and stirred for half an hour. The slurry was filtered using a suction filter, washed with ice water (2× with 200 ml) and dried at about 65° C. under vacuum for three days to yield Tolterodine hydrobromide (164.4 g) of 99.26% purity as determined by HPLC.

### Example 2

### Preparation of Tolterodine Hydrobromide

A solution was formed by combining the fumarate salt of N,N-diisopropyl-[3-(2-methoxy-5-methylphenyl)-3-phenylpropyl]-amine of structural Formula II (50 g, 0.110 mol, HPLC purity of 99.56%) and HBr in acetic acid (33%, 125 ml) and stirring at 75° C. to 80° C. for 14 hours in a glass reactor. The solution was cooled to room temperature and ice water (2000 ml) was added, forming a slurry. The slurry was cooled to 5° C.±5° C. and stirred for half an hour. The slurry was filtered using a suction filter, washed with ice water (2× with 250 ml), and dried at 60° C. under vacuum for three days to yield Tolterodine hydrobromide (166.7 g) of 98.23% purity as determined by HPLC.

### Example 3

### Preparation of Tolterodine Hydrobromide

A solution was formed by combining the fumarate salt of N,N-diisopropyl-[3-(2-methoxy-5-methylphenyl)-3-phenylpropyl]-amine of structural Formula II (50 g, 0.110 mol, HPLC purity of 99.67%) and HBr in acetic acid (33%, 125 ml) and stirring at 75° C. to 80° C. for 14 hours in a glass reactor. The solution was cooled to room temperature and water (500 ml) was added, forming a slurry. The slurry was cooled to 5° C.±5° C. and stirred for about 24 hours. The slurry was filtered using a suction filter, washed with ice water (2× with 40 ml), and dried at 60° C. under vacuum for about 18 hours to yield Tolterodine hydrobromide (32.1 g) of 98.90% purity as determined by HPLC.

### Example 4

### Preparation of Tolterodine Tartrate

Tolterodine hydrobromide (100 g, 0.246 mol), ethyl acetate (2 L) and water (500 ml) were mixed at room temperature in a glass reactor, forming a mixture. The mixture was stirred rapidly while adding potassium hydroxide (50%, 300 ml). After stirring thoroughly for approximately 15-30 minutes, two clear homogeneous layers formed. The layers were separated, and an organic phase was obtained. The organic phase was washed with water (2× with 500ml).

L-tartaric acid (38.33 g) dissolved in ethanol (800 ml) was added rapidly in one portion to the organic phase, at room temperature, forming a slurry. The slurry was cooled to 0° C.±5° C. over 2 hours and maintained at this temperature for about 15 hours. The slurry was filtered using a suction filter, washed with cold ethanol (2× with 100 ml), and dried at about 60° C. under vacuum for about 10 hours, yielding Tolterodine tartarate (62.6 g). The Tolterodine tartrate was recrystallized from dry ethanol twice, yielding Tolterodine tartrate (41.2 g) of 99.84% purity as determined by HPLC.

### Example 5

### Preparation of Tolterodine Tartrate

Tolterodine hydrobromide (100 g, 0.246 mol), ethyl acetate (2 L) and water (500 ml) were mixed at room temperature in a glass reactor, forming a mixture. The mixture was stirred rapidly while adding potassium hydroxide (50%, 300 ml). After stirring thoroughly for approximately 30 minutes, two clear homogeneous layers formed. The layers were separated, and an organic phase was obtained. The organic phase was washed with water (2× with 500 ml).

L-tartaric acid (38.4 g) dissolved in ethanol (800 ml) was added to the organic phase rapidly in one portion, at room temperature, forming a slurry. The slurry was cooled to 0° C.±5° C. over about 1 hour and maintained at this temperature for about 4 hours. The slurry was filtered using a suction filter, washed with cold ethanol (2× with 100 ml), and dried at about 60° C. under vacuum for about 10 hours to yield Tolterodine tartrate (65.2 g). The Tolterodine tartrate was recrystallized from dry ethanol, yielding Tolterodine tartrate (41.8 g) of 99.97% purity as determined by HPLC.

### Example 6

### Preparation of Tolterodine Tartrate

Tolterodine hydrobromide (583 g, 1.434 mol), ethyl acetate (20 L) and water (5 L) were mixed at room temperature in a glass reactor, forming a mixture. The mixture was stirred rapidly while adding potassium hydroxide (50%, 1.5 L). After stirring thoroughly for approximately 30 minutes, two clear homogeneous layers formed. The layers were separated and an organic phase was obtained. The organic phase was washed with water (2× with 5 L).

L-tartaric acid (385 g) dissolved in ethanol (8 L) was added rapidly in one portion to the organic phase, at room temperature, forming a slurry. The slurry was cooled to 0° C.±5° C. over about 1 hour and maintained at this temperature for about 12 hours. The slurry was filtered using a suction filter, washed with cold ethanol (2× with 1 L), and dried at about 60° C. under vacuum for 3 hours to yield Tolterodine tartrate (310 g). The Tolterodine tartrate was recrystallized twice from dry ethanol, yielding Tolterodine tartrate (219 g) of 99.98% purity as determined by HPLC.

### Example 7

### Preparation of Tolterodine Tartrate

Tolterodine hydrobromide (20 g, 0.049 mol), ethyl acetate (400 ml) and water (100 ml) were mixed at room temperature in a glass reactor, forming a mixture. The mixture was stirred rapidly while adding potassium hydroxide (50%, 35 ml). After stirring thoroughly for approximately 30 minutes, two clear homogeneous layers formed. The layers were separated and an organic phase was obtained. The organic phase was washed with water (2× with 100 ml).

The organic phase was added to L-tartaric acid (7.7 g) dissolved in ethanol (160 ml) over about 30 minutes at room temperature, creating a slurry. The slurry was cooled to 0° C.±5° C. over about 2 hours and maintained at this temperature for about 4 hours. The slurry was filtered using a suction filter, washed with cold ethanol (2× with 20 ml), and dried at about 60° C. under vacuum for about 14 hours to yield Tolterodine tartrate (12.5 g).

The Tolterodine tartrate (8.5 g) was recrystallized twice from dry ethanol, yielding Tolterodine tartrate (6.0 g) of 99.98% purity as determined by HPLC.

### Example 8

### Preparation of Tolterodine Tartrate

Tolterodine hydrobromide (20 g, 0.049 mol), ethyl acetate (400 ml) and water (100 ml) were mixed at room temperature in a glass reactor, forming a mixture. The mixture was stirred rapidly while adding potassium hydroxide (50%, 35 ml). After stirring thoroughly for approximately 30 minutes, two clear homogeneous layers formed. The layers were separated and an organic phase was obtained. The organic phase was washed with water (2× with 100 ml).

L-tartaric acid (7.7 g) dissolved in ethanol (160 ml) was added to the organic phase over about 2.5 hours at 70° C., forming a slurry. The slurry was cooled to 0° C.±5° C. over about 3 hours and maintained at this temperature for about 14 hours. The slurry was filtered using a suction filter, washed with cold ethanol (2× with 20 ml), and dried at about 60° C. under vacuum for about 3 hours to yield Tolterodine tartrate (10.8 g).

The Tolterodine tartrate (6.8 g) was recrystallized twice from dry ethanol, yielding Tolterodine tartrate (4.7 g) of 99.98% purity as determined by HPLC.

### Example 9

### HPLC Analysis

The purity determinations were performed using the following parameters. The column was a Chromsep SS Spherisorb 3CN (100×4.6mm, 3 μm) and the eluent comprised two mixtures. Mixture A had acetonitrile and 0.02 M KH₂PO₄ buffer (pH: 5.0) in a ratio of 20:80. Mixture B had 0.02 M KH₂PO₄ buffer (pH: 5.0). The gradient and time is illustrated in the following table:

| Time [min] | Eluent A [%] | Eluent B [%] |
|---|---|---|
| 0.0 | 100 | 0 |
| 5.0 | 80 | 20 |
| 10.0 | 80 | 20 |
| 20.0 | 100 | 0 |

The flow rate was 2.0 ml/min, and the run time was 25 min. The column thermostat was set for 25° C. and the sample thermostat was set for 5° C. The detection wavelength was set at 215 nm. The diluent was acetonitrile: water in a 1:1 ratio by volume. The injection volume was 10 μl. The detection limit was 0.02% and the quantification limit was 0.05%. If necessary, minor modification of the flow rate was permitted.

Typical retention times and relative retention times were:

| | | |
|---|---|---|
| Tolterodine: | RT: ˜8 min | RRT: 1.00 |
| DIP Amine MDPA: | RT: ˜17 min | RRT: 2.04 |

What is claimed is:

1. A process for the for the preparation of Tolterodine base of formula III by 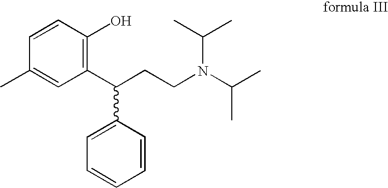
washing Tolterodine HBr with a base and a solvent selected from a group consisting of water, C₂₋₅ ester, C₂₋₆ ether, C₇₋₁₀ aromatic hydrocarbon and mixtures thereof.

2. A process for obtaining of R-Tolterodine tartrate of formula I 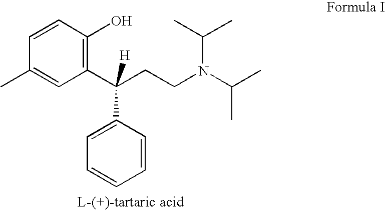
using a solution of L-tartaric acid in a C₁₋₄ alcohol and a solution of Tolterodine base of formula III in a solvent selected from a group consisting of water, C₂₋₅ ester, C₂₋₆ ether, C₇₋₁₀ aromatic hydrocarbon and mixtures thereof.

3. A process for the preparation of R-Tolterodine tartrate of formula I by cleaving the methyl ether of (N,N-diisopropyl-[3-(2-methoxy-5-methylphenyl)-3-phenylpropyl]-amine)fumarate of formula II 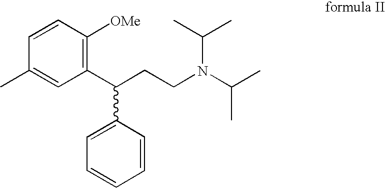
or salt thereof of formula IIa; 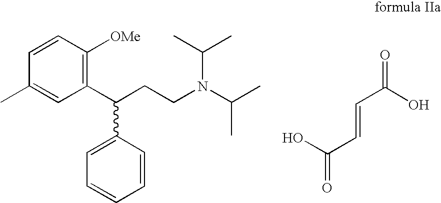
washing with a base and a solvent selected from a group consisting of water, C₂₋₅ ester, C₂₋₆ ether, C₇₋₁₀ aromatic hydrocarbon and mixtures thereof, performing an optical resolution; and crystallizing R-Tolterodine tartrate.

4. The process of claim 1, 2 or 3, wherein the C₂₋₅ ester is either ethyl acetate or isobutyl acetate.

5. The process of claim 4, wherein said C₂₋₅ ester is ethyl acetate.

6. The process of claim 1, 2 or 3, wherein the C₂₋₆ ether is diisopropylether.

7. The process of claim 1, 2 or 3, wherein the C₇₋₁₀ aromatic hydrocarbon is toluene.

8. The process of claim 1, wherein a mixture is obtained by combining Tolterodine HBr with a base and a solvent selected from a group consisting of water, C₂₋₅ ester, C₂₋₆ ether, C₇₋₁₀ aromatic hydrocarbon and mixtures thereof.

9. The process of claim 8, wherein the mixture is maintained at a temperature of about 15° C. to about 30° C. for about 15 to about 30 minutes.

10. The process of claim 9, wherein the mixture is maintained at a temperature of about 20° C. to about 25° C. for about 15 to about 30 minutes.

11. The process of claim 8, wherein the mixture is maintained while stirring.

12. The process of claim 1 or 3, wherein the base is either an inorganic or an organic base.

13. The process of claim 12, wherein the organic base is triethylamine, diisopropylethylamine, pyridine or morpholine.

14. The process of claim 12, wherein the inorganic base is added in a form of an aqueous solution.

15. The process of claim 12, wherein the inorganic base is either alkali hydroxide or alkali carbonate.

16. The process of claim 12, wherein the alkali hydroxide is either sodium hydroxide or potassium hydroxide.

17. The process of claim 12, wherein the alkali carbonate is either sodium carbonate or potassium carbonate.

18. The process of claim 12, wherein the base is potassium hydroxide.

19. The process of claim 1 or 3, wherein the base is added while stirring.

20. The process of claim 3, wherein R-Tolterodine of formula I is resolved without isolating the Tolterodine base of formula III.

21. The process according to claim 3, wherein the optical resolution comprises a solution of tartaric acid and a C₁₋₄ alcohol.

22. The process of claim 21, wherein the C₁₋₄ alcohol is either methanol or ethanol.

23. The process of claim 21, wherein said C₁₋₄ alcohol is ethanol.

24. The process of claim 21, wherein the L-tartaric acid solution is added into the Tolterodine base solution.

25. The process of claim 21, wherein the Tolterodine base solution is added to the L-tartaric acid solution.

26. The process of claim 25, wherein the L-tartaric acid solution is added all in one portion.

27. The process of claim 26, wherein the L-tartaric acid solution is added over a period of time.

28. The process of claim 27, wherein the L-tartaric acid solution is added over a period of less than about 3 hours.

29. The process of claim 2, wherein the solution of L-tartaric acid in a C₁₋₄ alcohol and a solution of Tolterodine base of formula III in a solvent selected from a group consisting of water, C₂₋₅ ester, C₂₋₆ ether, C₇₋₁₀ aromatic hydrocarbon and mixtures thereof, is combined at a temperature of about room temperature to about 70° C.

30. The process of claim 29, wherein combining the solutions leads to a slurry.

31. The process of claim 29 wherein the slurry is cooled to a temperature of about 5° C. to about −5° C. for about 5 to about 17 hours.

32. The process of claim 3, wherein it is run concurrently before the optical resolution.
